(19) United States
(12) Patent Application Publication
(10) Pub. No.: US 2023/0015994 A1
(43) Pub. Date: Jan. 19, 2023

(54) COMPOSITIONS AND METHODS FOR STYLING HAIR

(71) Applicant: L'Oreal, Paris (FR)

(72) Inventors: Vibha K. SHAH, Branchburg, NJ (US); Allison PERNER, Metuchen, NJ (US); Ronak RUGHANI, Edison, NJ (US); Brady ZARKET, Union, NJ (US); Sivaramakrishnan MUTHUKRISHNAN, Bridgewater, NJ (US); Aakash PAREKH, Edison, NJ (US)

(21) Appl. No.: 17/852,729

(22) Filed: Jun. 29, 2022

Related U.S. Application Data
(60) Provisional application No. 63/217,195, filed on Jun. 30, 2021.

(30) Foreign Application Priority Data
Sep. 20, 2021 (FR) 2109862

(51) Int. Cl. A61K 8/34; A61K 8/81; A61K 8/92; A61K 8/73; A61Q 5/06

(52) U.S. Cl. CPC A61K 8/347; A61K 8/8129; A61K 8/342; A61K 8/92; A61K 8/73; A61Q 5/06

(57) ABSTRACT

The disclosure relates to compositions comprising (a) at least one polyphenol; (b) at least one film former; (c) at least one emulsifier; (d) at least one fatty compound; and (e) at least one solvent. Optionally, the compositions may further comprise at least one oil, at least one humectant, and/or at least one fatty acid. The compositions can be used for styling hair, for example temporarily straightening the hair and/or for reducing frizz of hair. The disclosure also relates to methods of using the compositions to style hair, straighten hair, and/or reduce frizz of hair.

### TECHNICAL FIELD

The present disclosure relates to compositions and methods for styling hair, such as curly or wavy hair. The compositions provide temporary straightening properties to the hair. The disclosure also relates to methods of using the compositions to style the hair.

### BACKGROUND

Consumers with curly or wavy hair often desire to temporarily straighten their hair for a change in appearance. In addition, reducing the curls of very curly hair may increase the manageability and ease of styling of such hair.

Traditional hair-straightening products generally include heavy galenics or oils, which may leave an undesirable greasy or oily feeling. Products that provide hold, such as styling gels, can also give straightening effects, but these products typically flake on the hair. In addition, many hair straightening products involve heat or chemical treatments that may cause damage to the hair fibers and/or irritate the scalp.

As such, consumers desire new and improved compositions that can provide straightening effects to curly or wavy hair, and at the same time impart various additional advantageous properties to the hair such as softness, smoothness, shine, and/or frizz control.

It has now surprisingly been found that compositions comprising a particular combination of components as described herein are able to temporarily straighten curly or wavy hair without the use of heat or harsh chemicals, while at the same time delivering additional advantageous sensorial benefits to the hair.

### SUMMARY

The present disclosure relates to compositions for styling hair, such as curly or wavy hair. The compositions may provide beneficial effects such as straightening the hair, and/or one or more of softness, smoothness, shine, volume control, and/or frizz control to the hair. The compositions are optionally leave-in compositions, and may, in various embodiments, be in the form of a lotion or cream emulsion.

The hair styling compositions may comprise, in certain embodiments: (a) at least one polyphenol; (b) at least one film former; (c) at least one emulsifier; (d) at least one fatty alcohol; and (e) at least one solvent. Optionally, the hair styling compositions may further comprise at least one oil, at least one humectant, and/or at least one fatty acid.

The at least one polyphenol may be chosen from flavonoids and non-flavonoids, as well as mixtures thereof, and in some embodiments the polyphenol comprises, consists essentially of, or consists of flavonoids. In certain embodiments, the flavonoid comprises, consists essentially of, or consists of tannic acid. The at least one polyphenol may be present in an amount ranging from about 0.01% to about 10% by weight, relative to the total weight of the composition, such as, for example, about 0.1% to about 8%, about 0.5% to about 5%, or about 1% to about 3%. For example, the composition may in certain embodiments comprise tannic acid, present in an amount ranging from about 0.01% to about 10% by weight, relative to the total weight of the composition, such as, for example, about 0.1% to about 8%, about 0.5% to about 5%, or about 1% to about 3%.

The at least one film former may be chosen from, by way of example, carboxylic acid polymers, polysaccharides, gums, or mixtures thereof, and in at least some embodiments, comprises, consists essentially of, or consists of carrageenan. The at least one film former may be present in an amount ranging from about 0.01% to about 5% by weight, relative to the total weight of the composition, such as, for example, about 0.1% to about 5%, about 0.1% to about 3%, or about 0.1% to about 1%.

The at least one emulsifier may be chosen from, by way of example, crosslinked carboxylic acid polymers containing one or more monomers derived from acrylic acid, substituted acrylic acids, and salts and derivatives thereof, such as crosslinked homopolymers of an acrylic acid or of a derivative thereof, or crosslinked copolymers having (i) a first monomer selected from the group consisting of (meth)acrylic acid, derivatives thereof, short chain (e.g. C1-C4) acrylate ester monomers, or mixtures thereof, and (ii) a second monomer which is a long chain (e.g. C8-C40) substituted polyethylene glycol acrylate ester monomer. Optionally, the compositions comprise at least one co-emulsifier, for example at least one glyceryl ester. The at least one emulsifier (and co-emulsifier, if present) may be present in an amount ranging from about 0.01% to about 10%, such as from about 0.1% to about 8%, about 1% to about 6%, or about 2% to about 4% by weight, based on the total weight of the composition. In at least some embodiments, the emulsifier comprises Acrylamide/Sodium acryloyldimethyltaurate copolymer, isohexadecane, and Polysorbate 80, and optionally at least one co-emulsifier chosen from glyceryl esters.

The at least one fatty alcohol may, in various embodiments, be chosen from straight or branched chain, saturated or unsaturated fatty alcohols having from 8 to 30 carbon atoms, from 12 to 22 carbon atoms, or from 14 to 22 carbon atoms, derivatives thereof, or mixtures thereof, such as, for example decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof. The at least one fatty alcohol may be present in an amount ranging from about 0.01% to about 5% by weight, relative to the total weight of the composition, such as, for example, about 0.05% to about 4%, about 0.1% to about 3%, or about 0.5% to about 2%.

The solvent comprises water and optionally at least one non-aqueous solvent, and may be present in an amount ranging from about 50% to about 99%, such as about 60% to about 98%, or about 70% to about 95% by weight, relative to the total weight of the composition. In some embodiments, the solvent consists of, or consists essentially of, water, with the solvent comprising less than about 20%, less than about 10%, less than about 5%, or less than about 3% by weight, relative to the total weight of the composition, of non-aqueous solvents.

If present, the at least one oil may optionally be derived from a plant or vegetable, for example, oils from seeds or fruits. For example, the at least one oil may be chosen from hydrocarbon-based oil of plant origin and/or vegetable origin, or glyceride triesters, such as, for example, coconut oil, olive oil, jojoba oil, shea oil or shea butter, or argan oil. The oil may be present in an amount ranging from about 0.1% to about 20% by weight, relative to the total weight of the composition, such as, for example, about 0.5% to about 15%, about 1% to about 10%, or about 1% to about 6%.

If present, the at least one humectant may be chosen from, for example, glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof, or mixtures thereof. In certain embodiments, the composition comprises at least one humectant in an amount ranging from about 0.1% to about 10% by weight, relative to the total weight of the composition, such as, for example, from about 0.5% to about 8%, or about 1% to about 5%.

If present, the at least one fatty acid may be chosen from straight or branched chain fatty acids, which may be saturated or unsaturated, and may have, in various embodiments, from 8 to 30 carbon atoms, such as, for example, from 8 to 25, from 8 to 22, from 8 to 20, from 8 to 18, or from 8 to 16 carbon atoms, or from 12 to 30, from 12 to 25, from 12 to 22, from 12 to 20, or from 12 to 18 carbon atoms. The at least one fatty acid may be present in an amount ranging from about 0.01% to about 5% by weight, relative to the total weight of the composition, such as, for example, about 0.05% to about 4%, about 0.1% to about 3%, or about 0.5% to about 2%.

In some embodiments, the compositions further comprise at least one antioxidant, at least one preservative, at least one pH adjuster, at least one buffer, at least one vitamin, or mixtures of two or more thereof.

In some embodiments, hair styling compositions according to the disclosure may comprise (a) at least one flavonoid compound; (b) at least one film former chosen from polysaccharides; (c) at least one emulsifier; (d) at least one fatty alcohol; (e) water; (f) optionally, at least one oil; and (g) optionally, at least one humectant. The compositions may further comprise at least one fatty acid.

In some embodiments, hair styling compositions according to the disclosure may comprise (a) tannic acid; (b) at least one film former, optionally chosen from polysaccharides; (c) at least one emulsifier; (d) at least one fatty alcohol; (e) water; (f) optionally, at least one oil; and (g) optionally, at least one humectant. The compositions may further comprise at least one fatty acid.

In still further embodiments, hair styling compositions according to the disclosure may comprise (a) at least one polyphenol, optionally chosen from flavonoid compounds; (b) carrageenan; (c) at least one emulsifier; (d) at least one fatty alcohol; (e) water; (f) optionally, at least one oil; and (g) optionally, at least one humectant. The compositions may further comprise at least one fatty acid.

In certain embodiments, hair styling compositions according to the disclosure may comprise (a) tannic acid; (b) carrageenan; (c) at least one emulsifier, for example Acrylamide/Sodium acryloyldimethyltaurate copolymer, isohexadecane, and Polysorbate 80; (d) at least one fatty alcohol; (e) water, present in an amount of at least about 50% by weight, relative to the total weight of the composition; (f) optionally, at least one oil; and (g) optionally, at least one humectant. The compositions may further comprise at least one fatty acid.

In further embodiments, hair styling compositions according to the disclosure may comprise (a) tannic acid, present in an amount ranging from about 0.5% to about 6%; (b) carrageenan, present in an amount ranging from about 0.1% to about 2%; (c) at least one emulsifier comprising Acrylamide/Sodium acryloyldimethyltaurate copolymer, isohexadecane, and Polysorbate 80; (d) at least one fatty alcohol, present in an amount ranging from about 1% to about 5%; (e) water, present in an amount of at least about 60%; (f) at least one oil; and (g) at least one humectant, wherein all amounts are relative to the total weight of the composition. The compositions may further comprise at least one fatty acid.

The disclosure further relates to methods of using the compositions to style hair, such as methods of straightening hair, and/or methods of reducing frizz, wherein the methods comprise applying any one of the compositions described herein to wet, damp, or dry hair.

For example, in various embodiments, the disclosure relates to methods of styling the hair, methods of straightening the hair, and/or methods of reducing frizz of the hair, where the methods comprise applying to the hair a composition comprising: (a) at least one polyphenol; (b) at least one film former; (c) at least one emulsifier; (d) at least one fatty alcohol; and (e) at least one solvent comprising water. In various methods of styling, straightening, and/or reducing frizz of the hair, the at least one polyphenol is chosen from flavonoid compounds, for example tannic acid, which may be present in an amount ranging from about 0.01% to about 10%, such as, for example, about 0.1% to about 8%, about 0.5% to about 5%, or about 1% to about 3% by weight, relative to the total weight of the composition. In various embodiments, the at least one film former used in the compositions for styling, straightening, and/or reducing frizz of hair may be chosen from, for example, carboxylic acid polymers, polysaccharides, gums, or mixtures thereof, and in at least some embodiments is carrageenan, and may be present in an amount ranging from about 0.01% to about 5% by weight, relative to the total weight of the composition, such as, for example, about 0.1% to about 5%, about 0.1% to about 3%, or about 0.1% to about 1%. The at least one emulsifier, and optionally at least one co-emulsifier, in the composition useful in the methods may be present in an amount ranging from about 0.01% to about 10%, such as from about 0.1% to about 8%, about 1% to about 6%, or about 2% to about 4% by weight, based on the total weight of the composition. In at least some embodiments, the emulsifier comprises Acrylamide/Sodium acryloyldimethyltaurate copolymer, isohexadecane, and Polysorbate 80, and optionally at least one co-emulsifier chosen from glyceryl esters. The at least one fatty alcohol useful in the compositions used in the methods of styling, straightening, and/or reducing frizz of the hair may, in certain embodiments, be chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof, and may be present in an amount ranging from about 0.01% to about 5% by weight, relative to the total weight of the composition, such as, for example, about 0.05% to about 4%, about 0.1% to about 3%, or about 0.5% to about 2%. The solvent in the composition useful in the methods of styling, straightening, and/or reducing frizz of the hair comprises water and optionally at least one non-aqueous solvent, and may be present in an amount ranging from about 50% to about 99%, such as about 60% to about 98%, or about 70% to about 95% by weight, relative to the total weight of the composition. In various embodiments, the solvent consists of or consists essentially of water, with the solvent comprising less than about 10%, less than about 5%, or less than about 3% by weight, relative to the total weight of the composition, of non-aqueous solvents.

Optionally, the compositions useful in the methods of styling, straightening, and/or reducing frizz of the hair may further comprise at least one oil, at least one humectant, and/or at least one fatty acid. The at least one oil, if present, may optionally be derived from a plant, for example, oils from seeds or fruits, and may in certain embodiments be chosen from hydrocarbon-based oil of plant origin and/or vegetable origin, or glyceride triesters. If present, the at least one oil may be present in an amount ranging from about 0.1% to about 20% by weight, relative to the total weight of the composition, such as, for example, about 0.5% to about 15%, about 1% to about 10%, or about 1% to about 6%. If present, the at least one humectant compound in the methods of styling, straightening, and/or reducing frizz of hair may be present in an amount ranging from about 0.1% to about 10% by weight, relative to the total weight of the composition, such as, for example, from about 0.5% to about 8%, or about 1% to about 5%. The at least one fatty acid, if present in the compositions used in the methods for styling, straightening, and/or reducing frizz of the hair, may be chosen from straight or branched chain fatty acids, which may be saturated or unsaturated, and may have, in various embodiments, from 8 to 30 carbon atoms, such as, for example, from 8 to 25, from 8 to 22, from 8 to 20, from 8 to 18, or from 8 to 16 carbon atoms, or from 12 to 30, from 12 to 25, from 12 to 22, from 12 to 20, or from 12 to 18 carbon atoms, and may be present in an amount ranging from about 0.01% to about 5% by weight, relative to the total weight of the composition, such as, for example, about 0.05% to about 4%, about 0.1% to about 3%, or about 0.5% to about 2%.

Optionally, in order to style, straighten, and/or reduce frizz of the hair, any of the compositions disclosed herein may be left on the hair for a period of a few minutes to a few hours to a few days, for example until the hair is washed. In various embodiments, the compositions and/or methods do not include the use of a reducing agent, and/or do not include a step of heating the hair.

The compositions are not mascara compositions, and are not intended to be applied to the eyelashes or eyebrows. Similarly, the compositions are not make-up compositions, and are not intended to be applied to the skin.

### BRIEF DESCRIPTION OF THE FIGURES

The accompanying figures, which are incorporated herein and constitute part of this specification, illustrate exemplary embodiments of the disclosure, and, together with the general description given above and the description provided herein, serve to demonstrate features of the disclosure.

FIG. 1 is a photograph showing films formed from compositions 1B and C1, after drying.

FIG. 2 shows photographs of mannequin heads treated with compositions 1A, 1B, C1, and a control (water) while the hair is still wet (T0) and after the hair is dried (T1).

FIGS. 3A-3E are photographs showing volunteers' curly hair before treatment, and after treatment with compositions 1B-1E and C1, once the hair is dried.

It is to be understood that the foregoing and following descriptions are exemplary and explanatory only, and are not intended to be restrictive of any subject matter claimed.

### DETAILED DESCRIPTION

The disclosure relates to compositions for styling hair, for example compositions for straightening wavy or curly hair and/or reducing frizz of the hair, as well as to methods of using the compositions.

### I. Compositions

In various embodiments, compositions according to the disclosure comprise (a) at least one polyphenol; (b) at least one film former; (c) at least one emulsifier; (d) at least one fatty alcohol; and (e) at least one solvent. Optionally, the compositions may further comprise (f) at least one oil; (g) at least one humectant; and/or (h) at least one fatty acid. The compositions are optionally leave-in compositions, and may, in various embodiments, be in the form of a lotion or cream emulsion.

Compositions according to the disclosure may provide straightening effects to the hair, e.g. temporary straightening effects, and in at least some embodiments may provide one or more additional benefits such as softness, smoothness, shine, and/or frizz control to the hair. The compositions may, in various embodiments, be in the form of a lotion or a cream.

### Polyphenols

Compositions according to the disclosure comprise at least one polyphenol. Polyphenols are phenols with more than one phenolic —OH group that have the ability to act as “donor molecules” by donating their alcoholic hydrogen or accepting delocalized electrons. Useful polyphenols include both flavonoids and non-flavonoids.

Exemplary and non-limiting flavonoid compounds that can be used include, for example, flavanols, such as catechin, fisetin, kaempferol, myricetin, quercetin, rutin, proanthocyanidins, pyroanthocyanidins, theaflavins, or thearubigins (or thearubrins); dihydroflavonols, such as astilbin, dihydroquercetin, or silibinin; flavanones, such as hesperidin, neohesperidin, hesperetin, naringenin, naringin, or poncirin; flavones, such as apigenin, baicalin, diosmin, or rhoifolin; tannins, such as ellagitannins, tannic acid, gallic acid, or ellagic acid; isoflavonoids, such as biochanin A, Daidzein, or Genistein; fulvic acid, and neoflavonoids, as well as combinations thereof. In at least some embodiments, the polyphenol comprises, consists essentially of, or consists of flavonoid compounds.

Exemplary and non-limiting non-flavonoid compounds that can be used include, for example, stibenoids such as astringin, resveratrol, or rhaponticin, as well as combinations thereof.

Other polyphenols that can be used include hydroxycinnamic acids, for example, chlorogenic acid, verbascoside; phenolic aldehydes; phenylpropenes; coumarins, coumestans, or tyrosols, as well as combinations thereof. In one embodiment, the polyphenols may be plant-based and/or organic.

In certain exemplary embodiments, polyphenols useful according to the disclosure may be chosen from tannic acid, resveratrol, catechin, ellagic acid, resorcinol, gallic acid, humic acid, chlorogenic acid, quercetin, chebulinic acid, or mixtures thereof. In certain embodiments, the polyphenols are chosen from polyphenols that do not impart color to the hair. In at least some embodiments, the polyphenol comprises, consists essentially of, or consists of tannic acid.

In various embodiments, the at least one polyphenol is present in the compositions in an amount of about 0.01% to about 10% by weight, such as about 0.1% to about 8%, about 0.5% to about 7%, or about 1.5% to about 5.5% by weight, relative to the total weight of the composition. By way of non-limiting example, the at least one polyphenol is present in the composition with a total amount ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the composition, including all ranges and subranges thereof.

In some embodiments, the at least one polyphenol comprises, consists essentially of, or consists of tannic acid, where the total amount of tannic acid in the composition ranges from about 0.1% to about 8%, such as about 0.1% to about 7%, about 0.1% to about 6%, about 0.5% to about 6%, about 1% to about 6%, or about 2% to about 6% by weight, relative to the total weight of the composition, including all ranges and subranges thereof.

### Film Formers

Compositions according to the disclosure comprise at least one film forming agent, also referred to herein interchangeably as “film former.”

In various embodiments, the at least one film former may be chosen from, by way of example only, carboxylic acid polymers, polysaccharides, gums, or mixtures thereof.

By way of non-limiting example, the at least one film former may be chosen from carbomers, acrylates/C10-C30 alkyl acrylate crosspolymers, carrageenan, calcium carrageenan, potassium carrageenan, sodium carrageenan, or mixtures thereof. In various embodiments, the at least one film former is chosen from neutral acrylic homopolymers and copolymers, C1-C2 alkyl celluloses, C1-C3 alkyl guar, C1-C3 hydroxyalkyl guar, sclerotium gum, or mixtures thereof. In some embodiments, the film former does not comprise any homopolymers or copolymers of ethylene oxide, polyvinyl alcohols, homopolymers or copolymers of vinylpyrrolidone, homopolymers or copolymers of vinylcaprolactam, and/or homopolymers or copolymers of polyvinyl methyl ether. In further embodiments, the film former does not comprise a nonionic polymer other than those expressly disclosed herein. In still further embodiments, the compositions do not contain any film former other than carboxylic acid polymers, polysaccharides, gums, or mixtures thereof.

The at least one film former can be present in the composition in an amount ranging from about 0.01% to about 5%, such as from about 0.01% to about 4%, about 0.01% to about 3%, about 0.01% to about 2%, about 0.01% to about 1%, about 0.01% to about 0.8%, about 0.05% to about 5%, about 0.05% to about 4%, about 0.05% to about 3%, about 0.05% to about 2%, about 0.05% to about 1%, about 0.05% to about 0.8%, about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

In some embodiments, the at least one film former comprises, consists essentially of, or consists of carrageenan, and is present in the composition in an amount ranging from about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

### Emulsifiers

Compositions according to the disclosure comprise at least one emulsifier, which may in at least some embodiments comprise, consist essentially of, or consist of polymeric emulsifiers.

Non-limiting examples of emulsifiers that can be chosen include carboxylic acid polymers which are crosslinked compounds containing one or more monomers derived from acrylic acid, substituted acrylic acids, and salts and derivatives of these acrylic acids and substituted acrylic acids, such as crosslinked homopolymers of an acrylic acid or of a derivative thereof, e.g. acrylamidopropylsulfonic acid, or crosslinked copolymers having (i) a first monomer selected from the group consisting of (meth)acrylic acid, derivatives thereof, short chain (e.g. C1-C4) acrylate ester monomers, and mixtures thereof, and (ii) a second monomer which is a long chain (e.g. C8-C40) substituted polyethylene glycol acrylate ester monomer. Other non-limiting examples of emulsifiers include ethylene oxide/propylene oxide block copolymers; alkylpolyglucosides; phospholipids; ethoxylated esters of derivatives of natural oils such as the polyethoxylated ester of hydrogenated castor oil available commercially for example under the trade name Arlacel 989 (ICI); anionic emulsifiers such as fatty acid soaps e.g., potassium stearate and fatty acid sulphates e.g., sodium cetostearyl sulphate available commercially under the trade name Dehydag (Henkel); ethoxylated fatty alcohols, for example the emulsifiers available commercially under the trade name Brij (ICI); ethoxylated mono-, di-, and tri-glycerides, for example the emulsifiers available commercially under the trade name Labrafil (Alfa Chem.); methylglucose esters such as polyglycerol-3 methyl glucose distearate available commercially under the name Tegocare 450 (Degussa Goldschmidt); polyacrylamide emulsifier systems, acryloyldimethyltaurate polymeric emulsifiers such as those comprising acrylamide/sodium acryloyldimethyltaurate copolymer or mixtures thereof.

The at least one emulsifier may be present in the composition in an amount ranging from about 0.01% to about 15%, such as from about 0.1% to about 12%, about 1% to about 10%, about 2% to about 6%, or about 3% to about 5% by weight, based on the total weight of the composition, including all ranges and subranges thereof. For example, the at least one emulsifier may range from about 0.1% to about 9%, 0.1% to about 8%, 0.1% to about 7%, 0.1% to about 6%, 0.1% to about 5%, about 0.1% to about 4%, about 0.5% to about 10%, about 0.5% to about 9%, about 0.5% to about 8%, about 0.5% to about 7%, about 0.5% to about 6%, about 0.5% to about 5%, about 0.5% to about 4%, about 1% to about 10%, about 1% to about 9%, about 1% to about 8%, about 1% to about 7%, about 1% to about 6%, about 1% to about 5%, about 1% to about 4%, about 2% to about 5%, about 2% to about 4%, about 3% to about 5%, or about 3% to about 4% by weight, based on the total weight of the composition, including all ranges and subranges thereof.

In some embodiments, the at least one emulsifier is a polymeric emulsifier and is present in an amount ranging from about 1% to about 10%, about 1% to about 9%, about 1% to about 8%, about 1% to about 7%, about 1% to about 6%, about 1% to about 5%, about 1% to about 4%, about 2% to about 5%, about 2% to about 4%, about 3% to about 5%, or about 3% to about 4% by weight, based on the total weight of the composition, including all ranges and subranges thereof.

In one embodiment, the at least one polymeric emulsifier comprises a crosslinked anionic copolymer of acrylamide and of AMPS, e.g. in the form of a water-in-oil emulsion, such as those sold under the name SEPIGEL 305 (INCI name: Polyacrylamide/C13-14 Isoparaffin/Laureth-7), or an acryloyldimethyltaurate polymer such as Acrylamide/Sodium acryloyldimethyltaurate copolymer, sold under the name SIMULGEL 600, Acrylamide/Sodium acryloyldimethyltaurate copolymer/Isohexadecane/Polysorbate 80 by SEPPIC.

Compositions according to the disclosure may optionally further comprise at least one co-emulsifier, for example a co-emulsifier chosen from glyceryl esters (glycerol esters).

The glyceryl esters may, in some embodiments, have a carbon chain of 8 to 24 carbons. Non-limiting examples of glyceryl esters include bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl dioleate, glyceryl distearate, glyceryl laurate, or mixtures thereof.

In one exemplary and non-limiting embodiment, the emulsifier comprises Acrylamide/Sodium acryloyldimethyltaurate copolymer, isohexadecane, and Polysorbate 80, and optionally at least one co-emulsifier chosen from glyceryl esters.

### Fatty Alcohols

Compositions according to the disclosure comprise at least one fatty alcohol or fatty alcohol derivative.

Useful and non-limiting examples of fatty alcohols include those having from about 8 to about 30 carbon atoms, from about 12 to about 22 carbon atoms, and from about 14 to about 22 carbon atoms. The fatty alcohols can be straight or branched chain alcohols, and can be saturated or unsaturated. For example, the fatty alcohols may be chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof. In some cases, the fatty alcohols comprise at least one of or may be chosen from myristyl alcohol, cetyl alcohol, stearyl alcohol, cetearyl alcohol, isostearyl alcohol, oleyl alcohol, isotridecyl alcohol, derivatives thereof, or mixtures thereof.

The at least one fatty alcohol may be present in the composition in an amount ranging from about 0.01% to about 5% by weight, such as about 0.05% to about 4%, about 0.1% to about 3%, or about 0.5% to about 2% by weight, relative to the total weight of the composition. By way of non-limiting example, the at least one fatty alcohol is present in the composition with a total amount ranging from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, about 0.5% to about 5%, about 0.5% to about 4%, about 0.5% to about 3%, about 0.5% to about 2%, or about 0.5% to about 1% by weight, relative to the total weight of the composition, including all ranges and subranges thereof.

### Solvent

Compositions according to the disclosure comprise one or more cosmetically acceptable solvents. The solvent(s) may be chosen from water, non-aqueous solvents, or mixtures thereof. The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 95%, about 70% to about 90%, about 70% to about 85%, or about 75% to about 85% by weight, relative to the total weight of the composition.

In one embodiment, the solvent comprises water. In other embodiments, the composition comprises water and one or more non-aqueous solvents. For example, C₁-C₄ alcohols, organic solvents, polyols, glycols, hydrocarbons/alkanes, glycol ethers, or any mixture thereof. Non-limiting examples of solvents which may be used include alkane polyols such as, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In some exemplary embodiments, the solvent comprises water and at least one additional solvent chosen from linear or branched, saturated or unsaturated, and substituted or unsubstituted polyols, for example C₂-C₈ polyols or C₃-C₆ polyols, such as diols and/or triols. Any stereoisomer of the polyols may be used. In various exemplary embodiments, the C₂-C₈ polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In various embodiments, the solvent consists of, or consists essentially of, water. For example, in certain embodiments, the solvent comprises water in an amount ranging from about 50% to about 95%, about 60% to about 90%, about 70% to about 85%, or about 75% to about 80%, by weight of the solvent, with the remainder of the solvent comprising one or more additional solvents. For example, in certain embodiments, the composition comprises about 50% to about 99%, about 60% to about 98%, about 70% to about 95%, or about 75% to about 93% water, by weight of the composition. In other embodiments, the compositions comprise water and at least one non-aqueous solvent, where the total amount of non-aqueous solvent is present in an amount less than about 20%, such as less than about 18%, less than about 16%, less than about 15%, less than about 13%, less than about 12%, less than about 10%, less than 8%, less than 5%, or less than 3% by weight, relative to the total weight of the composition. In some embodiments, the compositions are free or substantially free of non-aqueous solvents.

### Oils

Compositions according to the disclosure optionally comprise at least one oil, which may, in some embodiments, be a low molecular weight oil. The oil may, in certain embodiments, be chosen from oils which are derived from a plant or vegetable, for example, oils from seeds or fruits.

Oils that may be used according to the disclosure include, by way of example, a hydrocarbon-based oils of plant origin and/or vegetable origin. Non-limiting examples of such oils include acai oil, almond oil, aloe vera oil, andiroba oil, annatto oil, avocado oil, argan oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, shea oil or shea butter, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

In some embodiments, oils that may be used according to the disclosure may include an oil chosen from glyceride triesters, which are generally triesters of fatty acids and of glycerol, the fatty acids of which can have varied chain lengths from C₄ to C₂₄, it being possible for these chains to be saturated or unsaturated and linear or branched. Non-limiting examples of these oils include wheat germ oil, sunflower oil, grape seed oil, sesame oil, corn oil, apricot oil, castor oil, shea oil, avocado oil, olive oil, soybean oil, sweet almond oil, palm oil (which contains palmitic acid), rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

If present, the total amount of the at least one oil that may be included in various embodiments ranges from about 0.01% to about 20%, such as from about 0.1% to about 20%, from about 1% to about 20%, or from about 2% to about 20% by weight, based on the total weight of the composition, including all ranges and subranges therebetween. In various embodiments, the oil may be present in an amount ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2% by weight, from about 2% to about 10%, from about 2% to about 9%, from about 2% to about 8%, from about 2% to about 7%, from about 2% to about 6%, from about 2% to about 5%, from about 2% to about 4%, or from about 2% to about 3% by weight, based on the total weight of the composition, including all ranges and subranges therebetween.

### Humectants

Compositions according to the disclosure optionally comprise at least one humectant. In certain embodiments, compositions according to the disclosure comprise at least two humectants.

Non-limiting humectant compounds that can be chosen include, for example, polyhydric alcohols, such as C2-C8 or C3-C6 polyhydric alcohols. By way of example, glycerol (glycerin), propylene glycol, butylene glycol, pentylene glycol, hexylene glycol, dipropylene glycol, diethylene glycol, and diglycerol may be chosen.

Additional non-limiting examples of humectants that can be chosen include hyaluronic acid, sugars such as sorbitol or xylitol, or beta-fructans, for example inulin. Mixtures of two or more humectants can also be chosen.

The humectants can be present in the composition in a total amount ranging from about 0.1% to about 10%, such as from about 0.5% to about 8%, or about 1% to about 5% by weight, based on the total weight of the composition, including all ranges and subranges thereof.

### Fatty Acids

Optionally, the hair styling compositions may comprise at least one fatty acid. For example, useful fatty acids include straight or branched chain fatty acids, which may be saturated or unsaturated. The fatty acids may, in some embodiments, have from 8 to 30 carbons, such as, for example, from 8 to 25, from 8 to 22, from 8 to 20, from 8 to 18, or from 8 to 16 carbons. In some cases, the fatty acid has from 12 to 30, from 12 to 25, from 12 to 22, from 12 to 20, or from 12 to 18 carbons. In further cases, the fatty acids may comprise C8-C22 or C14-C22 fatty groups, which may be saturated or unsaturated linear alkyl chains. The fatty acids may optionally have fatty groups chosen from stearate, isostearate, linoleate, oleate, behenate, arachidonate, palmitate, myristate, laurate, caprate, isostearyl, stearyl, linoleyl, oleyl, behenyl, myristyl, lauryl, or capryl chains, or mixtures thereof.

Non-limiting examples of fatty acids include diacids, triacids, and other multiple acids, as well as salts of these fatty acids. For example, the fatty acid may optionally include or be chosen from lauric acid, myristic acid, palmitic acid, stearic acid, behenic acid, archidonic acid, oleic acid, isostearic acid, sebacic acid, ricinoleic acid, 12-hydroxy stearic acid, or mixtures thereof. The fatty acids may, in some embodiments, be selected from the group consisting of palmitic acid, myristic acid, stearic acid, or mixtures thereof.

If present, the at least one fatty acid may be present in the composition in an amount ranging from about 0.01% to about 5% by weight, such as about 0.05% to about 4%, about 0.1% to about 3%, or about 0.5% to about 2% by weight, relative to the total weight of the composition. By way of non-limiting example, the at least one fatty acid is present in the composition with a total amount ranging from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, about 0.5% to about 5%, about 0.5% to about 4%, about 0.5% to about 3%, about 0.5% to about 2%, or about 0.5% to about 1% by weight, relative to the total weight of the composition, including all ranges and subranges thereof.

### Additional Components

Compositions according to the disclosure may optionally comprise any additional or auxiliary component suitable for use in such compositions. Such components may include, but are not limited to, conditioning agents (e.g. butters, such as, for example, shea butter), moisturizing agents, fillers, structuring agents, shine agents, antioxidants (e.g. tetradibutyl pentaerithrityl hydroxyhydrocinnamate, butylated hydroxyanisole, and/or butylated hydroxytoluene), penetrants, sequestrants, fragrances, buffers (e.g. citric acid and/or sodium citrate), dispersants, plant extracts, preserving agents (preservatives), opacifiers, sunscreen agents, pH adjusting agents, vitamins (e.g. tocopherol, ascorbic acid, and/or ascorbyl glucoside), and antistatic agents.

Optional additional or auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as from about 3 to about 6.8, from about 3.5 to about 6.5, from about 4 to about 6.5, from about 4.5 to about 6.5, from about 5 to about 6.5 or from about 5.5 to about 6.5. In some embodiments, the pH of the compositions may be substantially unchanged (e.g. may have a change in pH of less than 20%, less than 15%, or less than 10%) when stored under ambient conditions for 4 weeks or more, such as 8 weeks.

Compositions according to the disclosure are typically in the form of a lotion or cream, for example an oil-in-water (O/W) emulsion. In various embodiments, the compositions may have a viscosity ranging from about 1.75 Pa·s to about 3.75 Pa·s, such as about 2 Pa·s to about 3.6 Pa·s or about 2.5 Pa·s to about 3.4 Pa·s, when measured using a Rheomat RM 180, M3 spindle for 30 seconds under ambient conditions. In some embodiments, the viscosity of the compositions may be substantially unchanged (e.g. may have a change in viscosity of less than 20%, less than 15%, or less than 10%) when stored under ambient conditions for 3 days or more, such as 7 days or more, or 10 days or more.

### II. Methods

The present disclosure also relates to methods for styling hair, for example by temporarily straightening curly or wavy hair by application of the compositions described herein to the hair. The methods generally comprise applying any of the compositions according to the disclosure to the hair.

The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to temporarily straightening the hair, the compositions are surprisingly capable of providing a variety of desirable sensory benefits, for example, moisture, smoothness, softness, shine, and/or anti-frizz properties, to the hair. As such, the compositions are useful in styling (which includes straightening) hair while also caring for hair, conditioning hair, and/or imparting one or more above-described sensory benefits to the hair.

Typically, methods according to the disclosure may include applying an effective amount of a composition disclosed herein, e.g. cream emulsion, to hair having waves or curls, when the hair is dry, wet, damp, or moist. The hair may be either natural or synthetic hair. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. The composition may then be dried, for example air-dried or dried with a hair dryer. Heat may optionally be used during drying, but is not required. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer.

In some embodiments, before applying the composition to the hair, the hair may be first cleansed with a commercially available shampoo, and/or rinsed with water, and/or dried, either partially or completely. The composition may then be applied to the washed and/or rinsed hair when the hair is wet, damp, moist, or dry. In some other embodiments, before applying the composition to dry hair, the hair can optionally be moistened, damped, or wetted by water spray or using a wet towel, or by applying other treatment compositions that make the hair moist, damp, or wet.

It has surprisingly and unexpectedly been discovered that methods according to the disclosure generally do not require the use of a reducing agent, including a base, or heating the hair in order to achieve a temporary straightening effect. As such, in some embodiments, the method does not include using a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

The compositions described herein are not mascara compositions, i.e. are not intended to be applied to eyelashes and/or eyebrows. The compositions are likewise not makeup compositions, i.e. are not intended to be applied to skin, in particular, to skin on the face, body, or lips. Thus, treatment of, or application to, the hair is intended to be limited to hair on the head, i.e. the scalp of the head, and not eyelashes, eyebrows, body or facial hair, or the like. In such embodiments, a skilled person understands that “hair on the head” means hair that grows from the scalp of the head, and excludes eyebrows, eyelashes, facial hair, etc.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, or F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included. However, it is to be understood that such language also includes embodiments where G, H, I, etc., are present, as long as at least one of A, B, C, D, E, F, or a salt thereof, is also present.

The term “and/or” should be understood to include both the conjunctive and the disjunctive. For example, “A and/or B” means “A and B” as well as “A or B,” and expressly covers instances of either, without reference to the other. For example, a composition that comprises “at least one humectant and/or at least one fatty acid” means that the disclosure includes compositions comprising one or more humectants or one or more fatty acids, as well as compositions comprising one or more humectants and one or more fatty acids. As a further example, disclosure of “methods of styling the hair, straightening the hair, and/or reducing frizz of the hair” includes methods of styling the hair or methods of straightening the hair or methods of reducing frizz of the hair, as well as methods of styling the hair and straightening the hair, methods of styling the hair and reducing frizz of the hair, methods of styling the hair and straightening the hair and reducing frizz of the hair, etc.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Further, unless expressly stated otherwise or when the context of the disclosure clearly dictates otherwise, the language “the at least one X may be present in an amount ranging from Y % to Z %” is to be understood to mean that the total amount of X's present in the composition ranges from Y % to Z %, inclusive of the endpoints. For example, “the at least one polyphenol may be present in an amount ranging from about 0.01% to about 10%” means that the total amount of polyphenols present ranges from about 0.01% to about 10%, and is inclusive of both 0.01% and 10%.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of the hair on the head.

As used herein, the phrase “applying a composition onto hair” and variations thereof is intended to mean contacting the hair with at least one of the compositions of the disclosure, in any manner. It may also mean contacting the hair in an effective amount.

As used herein, the term “styling” is intended to include “shaping” and “straightening.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, unless expressly stated otherwise, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1—Compositions

The inventive and comparative compositions set forth in Table 1 below were prepared.

TABLE 1

| | Inventive | | | | | Comparative | |
|---|---|---|---|---|---|---|---|
| | 1A | 1B | 1C | 1D | 1E | C1 | C2 |
| SORBITAN OLEATE | 0.13 | 0.09 | 0.09 | 0.09 | 0.09 | | |
| TANNIC ACID | 5.0 | 5.0 | 3.0 | 3.0 | 3.0 | 5.0 | 5.0 |
| ACRYLAMIDE/SODIUM | 2.0 | 1.5 | 1.5 | 1.5 | 1.5 | | |
| ACRYLOYLDIMETHYL- | | | | | | | |
| TAURATE COPOLYMER | | | | | | | |
| ISOHEXADECANE | 1.0 | 0.79 | 0.79 | 0.79 | 0.79 | | |
| PROPYLENE GLYCOL | | | 2.0 | | | | |
| CETEARYL ALCOHOL | | 0.37 | 0.37 | 0.37 | 0.37 | | |
| DECYL GLUCOSIDE | | | | | | | 2.0 |
| BUTYROSPERMUM | | | | 2.0 | | | |
| PARKII (SHEA) BUTTER | | | | | | | |
| ETHYLHEXYL- | 0.09 | 0.09 | 0.09 | 0.09 | 0.09 | | 0.09 |
| GLYCERIN | | | | | | | |
| OLEA EUROPAEA | | | 1.0 | 1.0 | | | |
| (OLIVE) FRUIT OIL | | | | | | | |
| COCOS NUCIFERA | | 1.0 | 1.0 | 1.0 | 1.0 | | |
| (COCONUT) OIL | | | | | | | |
| GLYCERYL STEARATE | | 0.5 | 0.5 | 0.5 | 0.5 | | |
| CARRAGEENAN | 0.5 | 0.5 | 0.5 | 0.5 | 0.5 | 0.5 | 0.5 |
| POLYSORBATE 80 | 0.35 | 0.26 | 0.26 | 0.26 | 0.26 | | |
| ADDITIVES (Vitamins, | 0.9 | 0.9 | 0.9 | 0.9 | 0.9 | | 0.9 |
| preserving agents, pH | | | | | | | |
| adjusters, buffers) | | | | | | | |
| WATER | QS to | QS to | QS to | QS to | QS to | QS to | QS to |
| | 100 | 100 | 100 | 100 | 100 | 100 | 100 |

The compositions were prepared as follows. First, the solvent phase was heated to a temperature of about 70-75° C. The gum, polymer, and polyphenol components were added individually with mixing at medium speed using a rotor-stator blade on a Rayneri V2004 mixer. Once the polyphenol, polymers, and gum were fully dispersed, the rest of the ingredients were added one by one with mixing at medium speed to ensure each was fully incorporated, and an emulsion was formed. Then the batch was cooled to a temperature of about 30-35° C., while mixing with a propeller-type blade.

Each of compositions 1A-1E and C1-C2 provided temporary straightening benefits to hair, and reduced frizz even in high humidity. However, hair treated with compositions C1-C2 felt more stiff and coated than hair treated with compositions 1A-1E, which had a smoother, more natural feel.

### Example 2—Stability Study

In order to assess the stability of the inventive compositions relative to the comparative compositions, compositions 1B-1E and C1 were evaluated over time.

Compositions 1B-1E were studied for 10 days at 60° C. At the end of the study, no precipitation, phase separation, or change in pH was noted, and only a minor change in color was observed. Accordingly, compositions 1B-1E were considered stable.

Composition C1 was studied for 7 days at 60° C., and for 2 months at 45° C. At the end of both studies, composition C1 showed precipitation, demonstrated a color change from light brown to dark brown, and had a decrease in pH of approximately 1.2 pH units. Accordingly, composition C1 was considered less stable than compositions 1B-1E.

### Example 3—Film Quality

Films produced by compositions 1B and C1 were evaluated to determine film quality. Equal amounts of compositions 1B and C1 were weighed into identical weigh boats made of polyethylene terephthalate (PET), and allowed to completely dry in a dessicator.

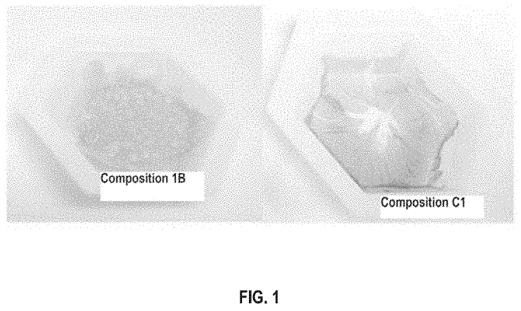

Once dry, the film formed by composition C1 was brittle and stiff, whereas the film formed by composition 1B was homogenous and not as brittle or stiff as the film of composition C1. Thus, the film formed by composition 1B was more favorable than that formed by composition C1. The dried films are shown in FIG. 1.

### Example 4A—In Vitro Testing on Hair

Compositions 1A, 1B, and C1, as well as water (control), were tested on mannequin heads with curly hair. First, the hair was washed and conditioned with a standard shampoo and conditioner, then excess water was removed with a towel and the hair was brushed. Next, equal amounts of composition 1A, 1B, C1, or water was applied to one-half of a mannequin head in an amount of 5 grams per half-head and the hair was combed, after which the hair was dried with a rollerball dryer at a temperature of about 40-45° C. for a period of about 60 minutes.

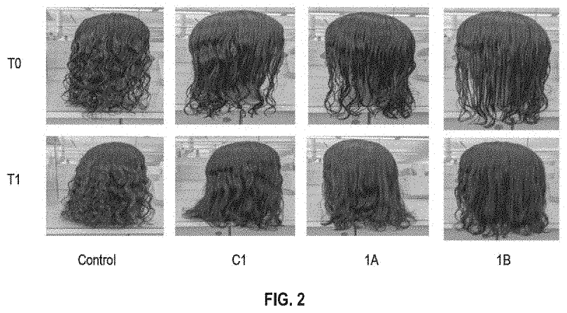

As can be seen in FIG. 2, hair treated with each of compositions 1A, 1B, and C1 provided straightening benefits to curly hair when wet (T0), and both straightening and frizz control to the hair once it had dried (T1).

### Example 4B—In Vivo Testing on Hair

Curly hair of several volunteers was washed and conditioned with standard shampoo and conditioner, and after excess water removed, composition 1B was applied to one half of the head of hair, and one of compositions C1, 1C, 1D, or 1E was applied to the other half of the head of hair, and the hair was combed. The hair was then dried with a rollerball dryer at a temperature of about 40-45° C. for a period of about 60 minutes. Table 2 below shows the specific compositions and amounts (in grams) used in each comparison.

TABLE 2
Consumer Testing

| FIG. | COMPARISON | C1 | 1B | 1C | 1D | 1E |
|---|---|---|---|---|---|---|
| 3A | Comparison 1 - frizzy, color- | 11.0 | 11.0 | | | |
| | treated hair with highlights | | | | | |
| 3B | Comparison 2 - curly, color- | 8.9 | 8.9 | | | |
| | treated hair | | | | | |
| 3C | Comparison 3 - curly, color- | | 5.0 | 5.0 | | |
| | treated hair | | | | | |
| 3D | Comparison 4 - wavy, color- | | 4.0 | | 4.0 | |
| | treated hair | | | | | |
| 3E | Comparison 5 - wavy hair | | 4.0 | | | 4.0 |

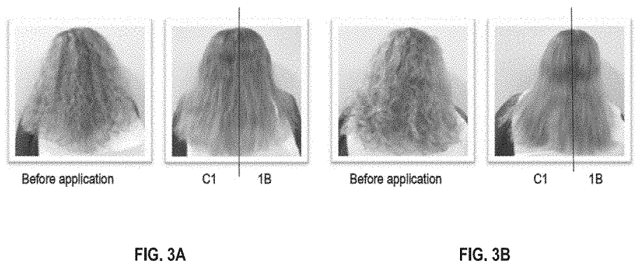

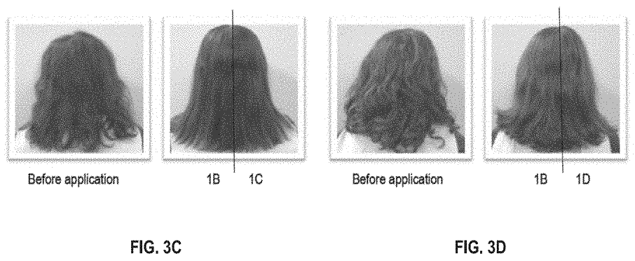

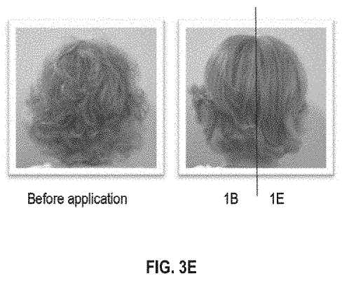

FIGS. 3A-3E show the hair before treatment and after the hair is dried (and before the film formed by composition C1 broken in FIGS. 3A-3B). As can be seen, the hair in each of the figures was noticeably straighter after treatment with each of compositions C1 and 1B-1E. However, the hair treated with compositions 1B-1E was smoother, less frizzy, and had a more natural feel.

The above Examples demonstrate that compositions according to the disclosure provide effective temporary straightening benefits to curly hair, while at the same time providing sensorial benefits such as smoothness and frizz-control to the hair. Further, compositions according to the disclosure have improved stability compared to compositions not according to the disclosure.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-29. (canceled)

30. A hair styling composition comprising:
(a) at least one polyphenol;
(b) at least one film former chosen from carboxylic acid polymers, polysaccharides, gums, or mixtures thereof;
(c) at least one emulsifier;
(d) at least one fatty alcohol;
(e) at least one solvent comprising water;
(f) optionally, at least one oil; and
(g) optionally, at least one humectant.

31. The hair styling composition of claim 30, wherein the total amount of polyphenols ranges from about 0.01% to about 10% by weight, relative to the total weight of the composition.

32. The hair styling composition of claim 30, wherein the at least one polyphenol comprises tannic acid.

33. The hair styling composition of claim 32, wherein composition comprises tannic acid in an amount ranging from about 0.1% to about 8% by weight, relative to the total weight of the composition.

34. The hair styling composition of claim 30, wherein the total amount of the film formers ranges from about 0.01% to about 5% by weight, relative to the total weight of the composition.

35. The hair styling composition of claim 30, wherein the at least one film former is chosen from polysaccharides.

36. The hair styling composition of claim 30, wherein the at least one film former comprises carrageenan.

37. The hair styling composition of claim 30, wherein the total amount of the emulsifiers ranges from about 0.01% to about 10% by weight, relative to the total weight of the composition.

38. The hair styling composition of claim 30, wherein the emulsifier comprises Acrylamide/Sodium acryloyldimethyltaurate copolymer, isohexadecane, and Polysorbate 80, and optionally at least one co-emulsifier chosen from glyceryl esters.

39. The hair styling composition of claim 30, wherein the at least one fatty alcohol is chosen from straight or branched chain, saturated or unsaturated fatty alcohols having from 8 to 30 carbon atoms, derivatives thereof, or mixtures thereof.

40. The hair styling composition of claim 30, wherein the at least one fatty alcohol is chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, fatty alcohol derivatives, or mixtures thereof.

41. The hair styling composition of claim 30, wherein the total amount of fatty alcohols ranges from about 0.01% to about 10% by weight, relative to the total weight of the composition.

42. The hair styling composition of claim 30, comprising at least one oil, wherein the total amount of oils ranges from about 2% to about 20% by weight, relative to the total weight of the composition.

43. The hair styling composition of claim 30, comprising at least one humectant chosen from glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof.

44. The hair styling composition of claim 30, comprising at least one humectant, wherein the total amount of humectants ranges from about 0.1% to about 10% by weight, relative to the total weight of the composition.

45. The hair styling composition of claim 30, wherein the solvent comprises water in an amount of at least about 50% by weight, relative to the total weight of the composition.

46. The hair styling composition of claim 30, wherein the solvent comprises water and at least one non-aqueous solvent, wherein the non-aqueous solvent is present in an amount less than about 20% by weight, relative to the total weight of the composition.

47. A hair styling composition comprising:
(a) tannic acid;
(b) carrageenan;
(c) at least one emulsifier comprising Acrylamide/Sodium acryloyldimethyltaurate copolymer, isohexadecane, and Polysorbate 80;
(d) at least one fatty alcohol;
(e) water, present in an amount of at least about 50% by weight, relative to the total weight of the hair styling composition;
(f) optionally, at least one oil of plant origin or vegetable origin; and
(g) optionally, at least one humectant.

48. A method for styling hair comprising applying to the hair a hair styling composition, wherein the hair styling composition comprises:
(a) at least one polyphenol;
(b) at least one film former chosen from carboxylic acid polymers, polysaccharides, gums, or mixtures thereof;
(c) at least one emulsifier;
(d) at least one fatty alcohol;
(e) at least one solvent comprising water;
(f) optionally, at least one oil; and
(g) optionally, at least one humectant.

49. The method of styling hair according to claim 48, wherein the composition does not comprise a reducing agent, and no step of heating the hair occurs after the composition is applied to the hair.
